(19) United States
(12) Patent Application Publication
(10) Pub. No.: US 2009/0149707 A1
(43) Pub. Date: Jun. 11, 2009

(54) ENDOSCOPIC BONE DEBRIDEMENT PORTAL

(76) Inventor: James K. Brannon, Leawood, KS (US)

Correspondence Address: INTELLECTUAL PROPERTY CENTER, LLC, 9233 WARD PARKWAY, SUITE 100, KANSAS CITY, MO, 64114, US

(21) Appl. No.: 12/369,575

(22) Filed: Feb. 11, 2009

Related U.S. Application Data
(63) Continuation-in-part of application No. 10/928,553, filed on Aug. 26, 2004, now Pat. No. 7,445,595.
Application No. 10/928,553 is a division of application No. 09/957,817, filed on Sep. 19, 2001, now abandoned.

(51) Int. Cl. A61B 1/00

(52) U.S. Cl. 600/114

(57) ABSTRACT

The present invention relates to an endoscopic bone debridement portal extending between an incision and a surgical site and adapted for receiving plural surgical tools and an endoscopic trephine, said endoscopic bone debridement portal comprising an elongated sleeve presenting a central channel and extending between a medial incision associated with the incision and a lateral surface associated with the surgical site, said elongated sleeve having plural guide surfaces for centrally aligning plural surgical tools and said central channel extending circumferentially around a lumen and between the plural guide surfaces for the alignment of surgical tools extending through the lumen from the incision towards the surgical site.

### CROSS REFERENCE TO RELATED APPLICATION

This application is a continuation in part of U.S. patent application Ser. No. 10/928,553 filed on Aug. 24, 2004 which is a divisional of U.S. patent application Ser. No. 10/957,817, filed Sep. 19, 2001, the contents of which are hereby incorporated by reference.

### FIELD OF THE INVENTION

This invention relates generally to endoscopic surgical instruments. Specifically, the present invention relates to a surgical portal that can receive plural surgical instruments and a method for using the same in orthopedic procedures.

### BACKGROUND OF THE INVENTION

1. Field of the Invention

Osteonecrosis of the femoral head in the young patient is a musculoskeletal disorder with growing concerns, particularly as osteolysis from particulate polyethylene wear debris compromises the longevity of a total hip arthroplasty. Approximately 20,000 new cases are reported each year, with an estimated 450,000 patients, on average, with ongoing disease in the United States. Lavernia et al. further reported in the Journal of the American Academy of Orthopaedic Surgeons in 1999 that osteonecrosis usually occurs during the prime of one's working years.

Osteonecrosis of the femoral head can be separated into two clinical categories, the symptomatic hip and the asymptomatic hip. Almost uniformly, 85% of symptomatic hips progress to collapse, irrespective of the stage of disease at the time of the initial diagnosis. It is often the asymptomatic hip wherein controversy arises regarding treatment. Urbaniak found in his series of asymptomatic patients that at least ⅔ would progress to collapse. Importantly, one may define impending collapse of the femoral head as greater than 50% head involvement in two radiographic orthogonal views. Bradway and Morrey, in the J of Arthroplasty 1993, found that a collection of 15 “presymptomatic”, hips all collapsed. Consequently, proponents of core decompression recommend early diagnosis and treatment of disease, with the understanding that such a treatment regimen may not halt progression.

Many theories have been proposed to explain the pathogenesis of osteonecrosis of the femoral head, as the name itself seems to describe the end condition, dead or nonviable osteocytes surrounded by a matrix of mineralized bone. More importantly, at least five categories have been identified as a potential mechanisms underlining the basis for disease: (1) Direct Cellular Mechanisms, cells die as a result of chemotherapy of thermal injury; (2) Extraosseous Arterial Mechanisms, ischemic necrosis of the femoral head following a substantially displaced fracture of the femoral neck; (3) Extraosseous Venous Mechanisms, an observation supported by the work of Ficat and Arlet in which these investigators observed venous hypertension in all clinical stages of osteonecrosis. Interestingly, The Johns Hopkins University observed compensatory mechanisms in the venous outflow of the femoral head when the venous system was obstructed using a dog model, raising questions about the role of venous congestion in the pathogenesis of disease; (4) Intraosseous Extravascular Mechanisms, this finding is thought to be consistent with bone marrow edema often observed on magnetic resonance imaging; and (5) Intraosseous Intravascular Mechanisms, occlusion of small vessels in patients with sickle-cell disease and dysbaric exposure wherein emboli of fat or nitrogen bubbles are thought to lead to osteneocrosis of the femoral head.

At lease four Stages of osteonecrosis are described to allow one to institute and compare various treatment regimens. The most frequently used staging system is that of Ficat and Arlet as follows: Stage I, normal plain film radiographs; Stage II, Sclerotic or cystic lesions without subchondral fracture; Stage III, Subchondral fracture (crescent sign), with or without articular incongruity; and Stage IV, osteoarthrosis with osteophytes. Other staging systems include those of Marcus et al., University of Pennsylvania System of Staging, Association Research Circulation Osseous (ARCO), and the Japanese Investigation Committee on Osteonecrosis wherein the location of the lesion determines the stage of disease.

At the histologic level, necrosis of the femoral head can be described as dead or nonviable to osteocytes surrounded by a mineralized matrix of bone. In a retrospective study by Marcus and Enneking, 13 core biopsies had been performed to treat eleven patients with asymptomatic or silent hips in Stage I or Stage II disease. All core biopsies in their series demonstrated normal articular cartilage, necrotic subchondral bone, and creeping substitution (osteoclastic bone resorption followed by the infiltration of marrow mesenchymal cells within a fibrovascular stroma). These observations and those of Phemister, Bonfiglio and others suggest that the success of core decompression in the treatment of osteonecrosis of the femoral head, Stage I or II, partly depends on the ability of autologous bone graft to incorporate the necrotic segment of bone within the femoral head. However, these authors did not attempt to characterize the requirements for host bone incorporation beyond an adequate blood supply.

The diagnosis of osteonecrosis can be easily made on plain film radiographs, assuming the disease is at least Ficat and Arlet Stage II, combined with a thorough history with an emphasis on predisposing risk factors, principally alcohol and steroid use, and a complete physical examination. Magnetic resonance imaging (MRI) may add additional information but is not routinely necessary. The MRI, however, is particularly useful in the asymptomatic hip, Ficat and Arlet Stage I.

Treatment options for ostonecrosis of the femoral head are categorized into one of two major groups, non-operative and operative. Nonoperatively, limited clinical success has been observed in the treatment of the symptomatic hip. Mont and Hungerford reviewed the nonoperative experience in the medical literature and found that only 22% of 819 hips in several pooled studies had a satisfactory result. These authors refer to the location of the osteonecrotic lesion, medial versus lateral, and suggest that medial lesions are more likely to have a satisfactory outcome. This observation is consistent with a mechanical component having a dominant role in the progression of disease, irrespective of etiology. Operative treatment can be characterized as core decompression of the femoral head with or without bone grafting followed by at least six weeks of non-weight bearing. Brown et al. at the University of Iowa used a three-dimensional finite-element model to elucidate the stress distribution over the diseased femoral head so as to characterize the optimal placement of a decompressing core with respect to location, depth, and diameter. More importantly, Brown et al. further showed that the optimum mechanical benefit of appropriately placed cortical bone grafts in a decompressed femoral head is realized when such grafts are situated in direct mechanical contact with the subchondral plate. These authors used the gait cycle to identify peak stress in the femoral head during normal walking and concluded that when fibula grafts are appropriately placed they potentially afford relief of stress to vulnerable necrotic cancellous bone in the subchondral and superocentral regions of the femoral head, implying that osseous incorporation of the cortical bone graft may be ideal but not completely necessary in the prevention of collapse. Although Brown et al. outlined the importance of strategic placement of a cortical fibula graft, it is important to recognize that these authors assumed that the necrotic cancellous bone is at risk for an intra-substance fracture, in the absence of treatment and that such intra-substance structural failure is principally responsible for the progression of disease, i.e., collapse of the femoral head. One must consider that as a segment of the femoral head becomes increasingly necrotic, its modulus of elasticity may vary substantially from that of the surrounding cancellous bone, and that progression of disease is perhaps also failure of this surrounding bone at the necrotic host bone interface; the area of creeping substitution in the work of Bonfiglio et al. Although not a part of the investigative objective, Brown et al. additionally did not demonstrate how cyclic loading of a cortical bone graft beneath the subchondral plate influences the healing behavior of the surrounding necrotic bone at the host necrotic bone interface. More specifically, is bone union achieved at the necrotic host bone interface now that the necrotic bone is unloaded? Is the fibula strut really a load-bearing cortical graft to the extent that the surrounding necrotic bone no longer sustains a substantial cyclic load during gait? Does the fibula strut simply allow the joint reactive force to bypass the segment of necrotic bone thereby substantially reducing its micromotion? Does micromotion of the necrotic segment of bone cause pain? Does the pain spectrum associated with osteonecrosis suggest a nonunion at the necrotic host bone interface, an intraosseous nonunion? These questions and others are prompted by the observation of good to excellent outcomes in patients with Ficat and Arlet Stage I or Stage II disease treated with core decompression with vascular and avascular cortical bone grafts, keeping aside the retrospective results of Kim et al. presented at the 1998 Annual Meeting comparing vascular to avascular fibula struts in treating osteonecrosis. More importantly, patients have been shown to benefit from core decompression alone implying that increased intraosseous pressure may play a dominant role in the early stages of disease, whereas in the later stages, the necrotic bone is less ductile and behaves in a more brittle fashion giving rise to subchondral collapse as evidence for a mechanical component playing a dominant role in the progression of disease. Recently, Mont et al. reported in the Journal of Bone and Joint Surgery good to excellent results in two groups of six dogs, twelve osteonecrotic hips, treated with trans-articular decompression of the femoral head and bone grafting, with and without osteogenic protein-1. Although the authors sought to elucidate the difference in healing time, i.e., the time to graft incorporation between the two groups, the critical observation is that all twelve hips were treated with avascular autograft. Therefore, Mont's work in view of Brown et al. causes one to consider the role a vascularized fibula graft in the treatment of osteonecrosis of the femoral head. Does revascularization really occur?

The work of Brown et al. suggests that core decompression is substantially core debridement of the femoral head. However, as one attempts to adequately debride the femoral head of osteonecrotic bone, the diameter of the core, by necessity, becomes increasingly large because one is not able to mechanically debride bone from the femoral head at a right angle to the decompressing core. Further, strategic placement of a cortical bone graft beneath the subchondral plate simply provides means for bypassing the at risk necrotic bone and transfers the load during gait to the fibula strut, which is often secured at the lateral cortex of the femur with a single Kirschner wire. The addition of a “blood supply,” vascularized fibula graft, in part relies on the work of Bonfiglio et al. Interestingly, actually “necrotic” autogenous bone stimulates osteoclastic bone resorption. In a recent issue of the Journal of Bone and Joint Surgery, Enneking showed in a histopathologic study that massive preserved human allografts (avascular bone) are slowly incorporated into host bone through limited bridging external callus and internal repair, even when rigid fixation is used to stabilize these grafts. Enneking suggests that the limited incorporation of allograft at cortical-cortical junctions could be enhanced with more recently developed osteoinductive substances. Importantly, however, is that Enneking observed enhanced bridging callus formation at allograft host junctions that were augmented with autogenous bone, and not increased internal repair that characterizes graft incorporation. Therefore, one is inclined to conclude that the newer osteoinductive substances may simply enhance external bridging callus and not internal repair. More importantly, Enneking observed bone at the allograft host junctions that lacked remodeling along the lines of stress. The critical issues here is that resorption must be followed by the infiltration of mesenchymal cells within a fibrovascular stroma for true incorporation to be established. Viable autograft appears to retain its ability to stimulate ongoing osteoclastic resorption whereas allograft lacks this ability, as it is principally osteoconductive. A blood supply may be more important at cortical-cortical junctions. Cortical-cancellous junctions depend on the nature of the host cancellous bone. Cortical bone will not incorporate necrotic cancellous bone as cortical bone lacks sufficient metabolic activity. However, given that cancellous bone is 8 times as metabolically active as cortical bone, one can expect incorporation of viable cortical bone at a cortical-cancellous junction. Within the growth plate, necrotic calcified cartilage stimulates osteoclastic resorption followed by the laying down of osteoid by osteoblast. In primary bone healing, osteoclast bore into necrotic segments of bone, which are then followed by the laying down of osteoid by osteoblast. One might recognize that in these examples, necrotic and avascular autogenous bone stimulates the infiltration of osteoclast and mesenchymal cells, and that external bridging callus in the presence of internal repair represents union. Enneking's work suggest that external bridging callus along human allograft bone is a surface event driven by local mesenchymal cells in the surrounding tissue while internal repair is limited as the cytokine germane to new bane formation within the allograft bone are lost during the sterilization process.

Einhorn et al. have shown that despite the great ingrowth of capillaries into fracture callus, the cell proliferation is such that the cells exist in a state of hypoxia. This hypoxic state could be favorable for bone formation, as in-vitro bone growth optimally occurs in a low-oxygen environment. Therefore, avascular autogenous bone in and of itself is not “bad” bone, Necrotic bone (a necrotic segment in the femoral head) retains its osteoinductivity and osteoconductivity. Osteoinduction is an avascular physiologic event dependent on BMP's, whereas osteoconduction is an avascular physical event dependent on the structural integrity of the inorganic extracellular matrix of bone. Urist in the Journal of Science in 1965 showed that “avascular” demineralized bone implanted in extra-skeletal sites would induce bone formation. Enneking has shown recently in the Journal that new bone formation can occur with massive allografts (necrotic bone) but internal repair (a physiologic event) is limited. More specifically, Hedrocel, a proprietary metal, will support the infiltration of osteoblast, with, the assumption that once infiltration is complete, new bone formation will ensue and ongoing remodeling (appositional new bone formation) will be sustained. Importantly, human allograft bone lacks osteoinduction sufficient to promote internal repair characteristic of bony union, as allograft bone is “processed” bone and consequentially may lose its ability to induce new bone formation. Necrotic or avascular autogenous bone retains its ability to induce and to conduct new bone formation, having a major requirement of stability and a healthy host bed. In this regard, as an osteoclastic front advances into the graft, avascular or necrotic, the mesenchymal cells that follow must continually receive the appropriate signals from cytokines (a physiologic event), and the graft must be sufficiently stable. Thus, one might consider the necrotic segment of the femoral head as a form of an unstable autograft and that the pathogenesis of osteonecrosis can be considered a mechanically unstable intra-osseus nonunion during the later stages of disease. An intra-osseous nonunion is to be distinctly differentiated from an extra-osseous nonunion wherein fibrous tissue characterizes the ununited bone. Clearly then, if stability of the necrotic segment of autogenous bone an be achieved, either through unloading of the necrotic bone or providing means for stabilization so as to facilitate internal repair where osteoinduction remains, union can be expected. The prevention of collapse and the absence of progression will characterize the extent and quality of union, i.e., internal repair.

To date, treatment modalities for osteonecrosis focus on attempts to deliver oxygenated blood to the necrotic bone within the femoral head. In a 1998 January/February article in the Journal of the American Academy of Orthopaedic Surgeons, Urbaniak describes a patent vascular pedicle using a fibula strut within a femoral head 5 days post-operatively. The patency of a typical vascularized fibula graft is usually assumed given the resolution of pain and the lack of progression of disease in a treated patient several years after the index surgery. The formal surgical procedure of decompression of the femoral head with vascularized fibula grafting usually requires prolonged surgery and is a demanding procedure. Vail and Urbaniak reported on donor site morbidity in 247 consecutive grafts in 198 patients at five years follow up. The authors observed an abnormality in 24% of limbs, a sensory deficit in 11.8% and 2.7% had motor weakness. Other complications reported by Urbaniak and Harvey in 822 vascularized fibula grafts procedures include superficial wound infections in two patients, and thromboembolic events in three patients.

Recently, Zimmer began an IDE study using a proprietary material, Hedrocel (trabecular metal, tantalum) as a mechanical device to fill a surgically created void in a femoral neck of a decompressed femoral head. The trabecular metal has a compressive and an elastic modulus similar to cancellous bone. The current IDE study is designed to evaluate the safety and efficacy of trabecular metal in the treatment of patients with early stage disease. The frictional properties of trabecular metal interfaced against cancellous bone are outlined as means for securing the implant within host bone. More importantly, the current investigation is of a nature thought to promote revascularization of the femoral head. Trabecular meal is osteoconductive and promotes bony ingrowth. In this regard, bony ingrowth is unidirectional growth, i.e., growth from the surrounding bone into the trabecular metal implant. As an aside, Zimmer promotes an acetabular component in which trabecular metal overlies the outer surface of the component. Bony ingrowth is promoted along the surface of the implant as means for establishing its stable fixation. In this setting, unidirectional growth is ideal, i.e., bony ingrowth into the implant. However, trabecular metal or any synthetic component juxtaposed necrotic bone will not promote new bone formation in a direction away from the implanted device and toward the necrotic bone. Further, such a large porous material will create a physiologic demand on bone formation in a direction away from the necrotic bone toward and into the implanted device, when in fact the purpose of treatment, particularly vascularized fibula grafts, is to direct bony ingrowth into the necrotic bone, i.e., bone growth in a direction away from the fibula strut and into the necrotic bone. More specifically, an acetabular component with trabecular metal on its outer surface has clinical value, whereas a mechanically stable column of trabecular metal within the femoral neck of a patient with osteonecrosis has less than obvious clinical value, as the bony ingrowth in this setting is in a direction away from the necrotic bone, thereby almost ensuring that the necrotic bone will not undergo sufficient internal repair as characterized by Enneking. One might surmise that complete debridement of the femoral head of necrotic bone and subsequent stabilization with trabecular metal may very well serve the clinical objectives of operative treatment. However, it is more prudent to stabilize the femoral head with autogenous cancellous bone. More succinctly, why discard a column of cancellous bone antecedent to the segment of necrotic bone within the femoral head? The antecedent cancellous bone is viable and is useful clinically. Clearly then, successful incorporation of a necrotic segment of bone requires bidirectional bony ingrowth. Bi-directional bony ingrowth is only available with viable cancellous autograft.

With the understanding as outlined, it is desirable to provide a device and method for adequately visualizing and accessing an internal region for debriding a femoral head of necrotic bone, while replacing the necrotic bone with viable cancellous bone, and providing support to a region of overlying cartilage. It is the purpose of the invention described herein to achieve these objectives using a novel device and a minimally invasive surgical technique.

### 2. Information Disclosure Statement

Bone grafting is among one of the most frequently performed surgical procedures by surgeons challenged with reconstructing or replacing skeletal defects. Over the years, several techniques have been devised to obtain and implant autologous bone. Scientist and clinicians have sought and defined the essential elements of bone healing and have further desired to secure these elements when considering the benefits of various types of bone grafting techniques. Recently, scientific inquiry has been directed toward understanding the role of bone morphogenic protein (BMP) in the process of new bone formation. What we have learned is that a simple fracture incites a tremendous cascade of events that lead to new bone formation, and that reducing this cascade to a product that can be sold is a difficult task if not impossible. Nonetheless, complex fractures continue to occur which orthopedic surgeons manage daily. Therefore, if one is to appreciate the invention at hand the essentials of fracture healing and new bone formation must be understood.

The essential elements required for bone regeneration are osteoconduction, osteoinduction, and osteogenic cells. In. this regard, autogenous bone is the gold standard for bone harvesting. Cancellous bone, as does cortical bone, contains all of these elements but lacks structural integrity. Cortical bone has structural integrity but is limited in quantity. At the histologic level, cortical bone is 4 times as dense as cancellous bone, and cancellous bone is 8 times as metabolically active as cortical bone. Further, clinicians have recognized the consequences of donor site morbidity and prolonged hospitalization after a traditional harvesting technique. To circumvent some of these issue, numerous synthetic bone like products have been made available for general use. Each product. attempts to exploit one or more of the three essential elements of bone regeneration described above. Although many of these products, e.g., Pro Osteon, INTERPORE, Collagraft, ZIMMER and others are unique, they remain expensive.

To define a less invasive technique for bone harvesting, percutaneous methods have been described. The recently developed techniques simply involve using a coring cylindrical device to obtain a segment of bone. David Billmire, M.D. describes this technique in his article, Use of the CORB Needle Biopsy for the Harvesting of Iliac Crest Bone Graft, PLASTIC AND RECONSTRUCTIVE SURGERY, February 1994. Billmire makes no effort to ensure the quality of the harvested bone but rather describes a power-driven counter-rotating hollow needle as cutting through bone and soft tissue. Michael Saleh describes a percutaneous technique for bone harvesting in his article, Bone, Graft Harvesting: A percutaneous Technique, Journal of Bone and Joint Surgery [Br] 1991; 73-B: 867-8. The author describes using a trephine to twist and lever out a core of bone of 8 mm in size. INNOVASIVE DEVICES describes using their COR™ System for arthroscopic bone graft harvesting. This system describes a disposable cutter having a distal cutting tooth projected into the lumen of the Harvester. This cutting tooth ensures that all harvested osteochondral bone grafts will have a uniform dimension. This cutting tool also serves as means for removing the harvested bone from its donor site. Further, the plunger of the COR™ System is used to disengage gently the harvested bone so as to maintain the overall length of the graft. This concept is absolutely essential to the successful use of the COR™ System as these precisely obtained samples of osteochondral bone are implanted into pre-drilled osteochondral defects within the knee. Further a vacuum of any sort could not be used on the COR™ System, as the vacuum would simply continue to extract water from the knee joint thereby failing to create an effective pressure drop across the harvested bone and loss of operative visualization. Brannon, in U.S. Pat. No. 6,007,496 describes the use of a vacuum apparatus to create a pressure drop across an osteopiston of bone. Scarborough et al., in U.S. Pat. No. 5,632,747 described a device for cutting short segment dowels from a bone mass.

When considering bone for grafting purposes, the recipient site must be considered as well. Failure to achieve bony union at a fracture site or bony fusion at a fusion site may be caused by several factors. Often, the blood flow is inadequate at the fracture site because of local trauma during the inciting event, as might be the case in osteonecrosis of the femoral head. Further, when considering augmentation of the healing process with bone graft, it is imperative that the grafted bone contains all of the essential elements germane to successful osseous regeneration, namely, osteoconductive elements, osteoinductive elements, and osteoprogenitor cells. Most current devices used for bone grafting focus on quantity, the osteoconductive portion of the harvested bone, and less so on quality, the osteoinductive portion of the harvested bone. Recently, bone substitutes have been developed and can be classified according to the following major categories: 1) Osteoconductive synthetics (Pro Osteon 500), 2) Osteoinductive allograft (Grafton), 3) Osteoinductive biosynthetics (OP-1), 4) Osteoinductive autologous bone marrow aspirates, 5) Osteoconductive/Osteoinductive combination synthetics, and 6) Gene therapy. When implanting the above bone graft substitutes, recognizing the usefulness of a collection of bone growth elements at the fracture site or those generated during the process of open reduction and internal fixation (ORIF) or any other bony procedure, such as posterior spinal instrumentation, has not been achieved through the development of a simple device to promote in situ bone grafting. In this regard, synthetic alternatives to bone grafting can be used as expanders that can be added to autogenous bone and mesenchymal cells harvested in situ at the fracture site or the surgical site. This approach will indeed ensure that all patients are given an optimal opportunity for bony union or bony fusion.

To recognize the issues at hand governing the invention described herein, a simple discussion of biomechanics, physiology, and general physics is warranted and presented in support hereof.

Bone is a viscoelastic material, and as such, it behaves predictably along its stress strain curve when axially loaded in either tension or compression. The key word here is viscoelastic. The prefix “visco” describes the fluid component of the material being tested and the suffix “elastic” describes the recoil potential of the material being tested. The ratio of stress: strain is Young's Modulus. Clearly, a spring is fully elastic. One may place a tension force on a spring, but when the tension is released, the spring recoils to its original length. A syringe, on the other hand, with a thin hypodermic needle attached, is considered viscoelastic. In other words, the amount of deformation observed is time dependent. Simply, the deformation will remain after the tension is removed. Consider one throwing Silly Putty against the ground and observing it bounce versus letting the material sit on a counter for several hours. One should appreciate that minimal deformation occurs when the Silly Putty bounces from the floor versus sitting it on a counter for several hours. The deformation is time dependent because of the internal fluid properties of the material; an amount of time is required to observe a net fluid flow. Bone behaves in a similar fashion, but has the additional property of being able to respond to a given stress by forming new bone. When bone fails to respond favorably, it fractures.

The physiologic properties of bone hinge on the fluid elements that govern bone regeneration, namely, bone morphogenic protein, various horomones, and osteoprogenitor cells. These fluid elements are important to the physiologic function of bone and are found within the bone marrow and the circulatory system. Appreciate that there is a net flow of these elements as bone bares a daily physiologic load during normal walking. Since the circulatory system is a closed system, a net loss of these fluid elements is not observed but rather continuous remodeling of bone and metabolic maintenance of the various cells and proteins as they age and become nonfunctional. Bone is incompressible above or below its elastic limit, i.e., Young's Modulus. Poisson's ratio is used to describe this behavior and is defined as follows:

v=−(delta d/do)/(delta 1/10) (1).

Poisson's ratio can be thought of as a measure of how much a material thins when it is stretched, consider taffy, or how much a material bulges when it is compressed. Regarding bone, one does not necessarily observe an increase in volume when it is compressed, but rather an increase in the density as bone remodels along the lines of stress, i.e., form follows function, Wolf's Law. When bone is compressed beyond its elastic limit, it fractures, i.e., it expands, therefore, its area will increase in a direction perpendicular to the line of force. The fracture observed occurs in the osteoconductive portion of bone, and a fluid flow will occur, as a result of the fracture, within the osteoinductive portion of bone.

The physiology of bone form and function is clear, but what a physician may observe through a series of x-rays may vary from patient to patient. Clearly then what we look for on a x-ray is evidence of healing, and in this regard, fracture healing is divided into at least four categories as follows: 1) inflammatory stage, 2) soft callus stage, 3) hard callous stage, and 4) remodeling stage. Each of these stages has clinical parameters that can be evaluated at the bedside. It is important to note, however, that any healing process in the human begins with clot formation; consider a simple laceration. Thus, fracture healing begins with clot formation. However, this stage of fracture healing does not have a clinical parameter unless the fracture is considered an open fracture and the absence of bleeding is observed.

The continues fluid nature of whole blood (formed elements, i.e., blood cells; serine proteases, i.e., clotting factors; proteins, carbohydrates, electrolytes and hormones) while circulating in the vascular system is substantially maintained by the endothelial lining along with the vessel walls. When these circulating serine proteases are exposed to subendothelial collagen or surfaces other than endothelial cells, i.e., abnormal surfaces, platelets aggregate and the clotting cascade is initiated. Blood without formed elements is considered plasma, while plasma without clotting factors is considered serum. A collection of autogenous bone growth elements is considered any and all factors germane to bone formation.

The clotting cascade is divided into two arms; the intrinsic pathway, i.e., local tissue trauma incites clot formation through exposure of the subendothelial collagen to circulating serine proteases and platelets; and the extrinsic pathway which incites clot formation through the activation of Factor VII serine protease and by tissue thromboplastin released from damaged cells. Both pathways then converge on Factor X serine protease. Regarding platelets, these cells are first to arrive and become adherent to injured tissue and form a platelet plug. Adherent platelets are activated platelets and as such release hemostatic agonist and autologous growth factors through a process of degranulation. The hemostatic agonists promote clot formation to ensure that the bleeding stops, while the autologous growth factors initiate the healing process of the injured tissue. Unique to bone is that its healing process is more regenerative of new bone formation as opposed to reparative which is more indicative of scar formation. Scar formation in fracture healing is a nonunion. Further, when bone fractures as a result of surgical or unintentional trauma, a collection of bone growth elements are generated directly within the fracture that contain both fluid and non-fluid components. Within the fluid component are platelets, blood and bone marrow mesenchymal cells, collagen and noncollagenous proteins, and small spicules of bone. The solid components is considered the bony fragments. ORIF is specifically designed to restore length and alignment of the fractured bone through rigid fixation of the non-fluid component. Bone grafting is used when it is determined preoperatively that the structural integrity and the quantity of the bony fragments are insufficient to allow ORIF. Clearly, the collection of bone growth elements required for bony union are present at the fracture site at the time of surgical (core decompression) or unintentional trauma. It stands to reason that in situ autologous bone growth elements, fluid and non-fluid, should be retained and used in conjunction with means for stabilizing the intra-osseous nonunion within the osteonecrotic femoral head. In situ autologous bone growth factors at a given fracture site unequivocally include the approximate level of BMP's and other noncollagenous proteins at the various stages of fracture healing as described above. Understanding the physiology of new bone formation, a reparative process, will lend credence to how one should collect and use bone graft elements harvested in situ or from a second operative site.

In addition, endoscopy is widely used to effect removal of the unwanted or damaged tissues from a patient in a manner that is less invasive than completely opening up the tissue and using traditional tools, resulting in a greatly shortened patient recovery, minimal scarring, reduced costs, elimination of typical pre-operative and post-operative hospital stays and widespread use to repair, replace or correct injuries or various orthopedic structures.

Generally, prior endoscopes allow a doctor to directly view the surgical site through an incision to observe, diagnosis or treat the patient. Typical endoscopes include a magnifying lens and coated glass fibers that beam intense, cool light into the surgical site while allowing observation and work on the site through multiple incisions. An endoscopic video camera is typically used for observing the surgical site through one of the incisions. Using an endoscopic video camera, the surgeon typically uses a separate incision for inserting a separate surgical instrument, such as mechanical and electrical cutting and cauterizing tools, shavers or other well known instruments. In addition, depending on the surgical procedure a number of devices must be utilized requiring multiple incisions or removal and insertion of the surgical device through relatively small incisions to the surgical site. For example, if the view of a surgical site has become obscured through excessive bleeding the surgeon may need to remove the cutting instrument and then insert an electronic device for cauterizing the bleeding tissue and blood vessels. Then the site may need to be evacuated using a suction instrument to restore vision to the surgical site. This insertion and removal procedure can go on for protracted periods until the desired surgical affect is achieved.

While the various known surgical tools are useful in performing a variety of surgical functions, they usually require plural incisions with multiple removal and insertion techniques to observe and perform the surgical operation causing the surgery to be unduly complicated and protracted. In addition, some prior art procedures utilize portals, few if any of such prior art procedures, or prior art devices used to perform such procedures effectively facilitate debriding, treating, repairing or replacing an orthopedic bone section.

It therefore would be beneficial to provide an efficient surgical instrument to facilitate endoscopic treatment of a patient's orthopedic material while providing access through a portal for insertion and retraction of surgical instruments at the surgical site, facilitating simultaneous surgical functions at a single location for removing, repairing and treating both hard and soft tissues.

### SUMMARY OF THE INVENTION

The present invention reduces the difficulties and disadvantages of the prior art by providing an endoscopic bone debridement portal extending between an incision and a surgical site, said endoscopic bone debridement portal comprising an elongated sleeve presenting a central channel and extending between a medial incision associated with the incision and a lateral surface associated with the surgical site, said elongated sleeve having plural guide surfaces for centrally aligning plural surgical tools and said central channel extending circumferentially around a lumen and between the plural guide surfaces for the alignment of surgical tools extending through the lumen from the incision towards the surgical site. In addition, the present invention includes the endoscopic bone debridement portal in combination with an endoscopic trephine and plural surgical instruments extending between an incision and a surgical site, the combination including plural surgical instruments having an operational head extending from a leader, said operational head adapted for surgical procedures, said endoscopic trephine having a distal endoscopic end, a proximal handle end, an inner visual surface and an outer contact surface, said proximal handle opposite said distal endoscopic end and said inner visual surface associated with said distal endoscopic end along said outer contact surface, the endoscopic portal having an inner surface and an outer surface presenting a seal at the juncture of the surrounding surface and the endoscopic portal, said inner surface adapted for sealable receipt of the outer contact surface, said distal endoscopic end adapted for passage through the endoscopic portal whereby said leaders are positioned longitudinally through an incision opening adapted for receiving said portal.

### BRIEF DESCRIPTION OF THE DRAWINGS

FIG. 1 is a perspective view of a prior art surgical procedure.

FIG. 1A is a sectional anteriorposterior view of a typical femoral head and neck.

FIG. 1B is a sectional lateral view of femoral head and neck.

FIG. 2 is a cross-section side view of the endoscopic bone debridement portal associated with the femoral head and neck of FIGS. 1A and 1B according to the present invention.

FIG. 2B is a geometric view of an autogenous cancellous osteomedullary bone cylinder.

FIG. 4A is a cross-section side view of the endoscopic bone debridement portal.

FIG. 5A is a right perspective view of the endoscopic portal in receipt of a surgical tool as illustrated in FIG. 2.

FIG. 5B is a right side perspective view of the endoscopic portal according to FIG. 5A.

FIG. 5C is a top plan view of the endoscopic portal illustrated in FIG. 5A.

FIG. 5D is a side elevational view of the endoscopic portal illustrated in FIG. 5A.

FIG. 5E is a cross-sectional view of the endoscopic portal illustrated in FIG. 5A.

FIG. 5F is a front view of the endoscopic portal illustrated in FIG. 5A.

FIG. 6 is a cross-sectional view of the femoral head receiving an osteoendoscopic cylinder through the endoscopic bone debridement portal.

FIG. 7 is a sectional view of the ostendoscopic cylinder of FIG. 6 during debridement.

FIG. 8 is a magnified sectional view of the femoral head from FIG. 7.

FIG. 10A is a side elevation view of the osteoendoscopic cylinder.

FIG. 10B is a top plan view of the osteoendoscopic cylinder.

FIG. 11A is a sectional anteriorposterior view of the proximal femur.

FIG. 11B is a sectional anteriorposterior view of the proximal femur.

FIG. 11C is a cross sectional view of the cancellous osteomedullary bone cylinder.

FIG. 11D is a sectional view of the treated femur.

### DETAILED DESCRIPTION OF THE INVENTION

### I. Introduction.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

### II. Endoscopic Bone Debridement Portal.

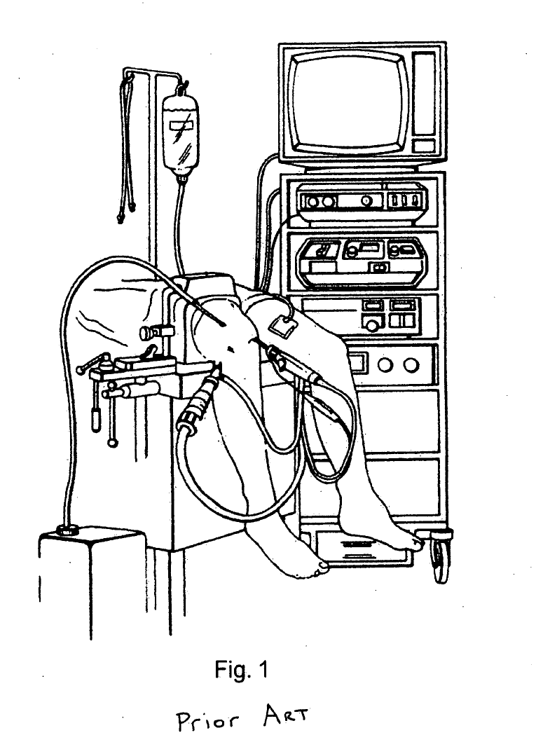

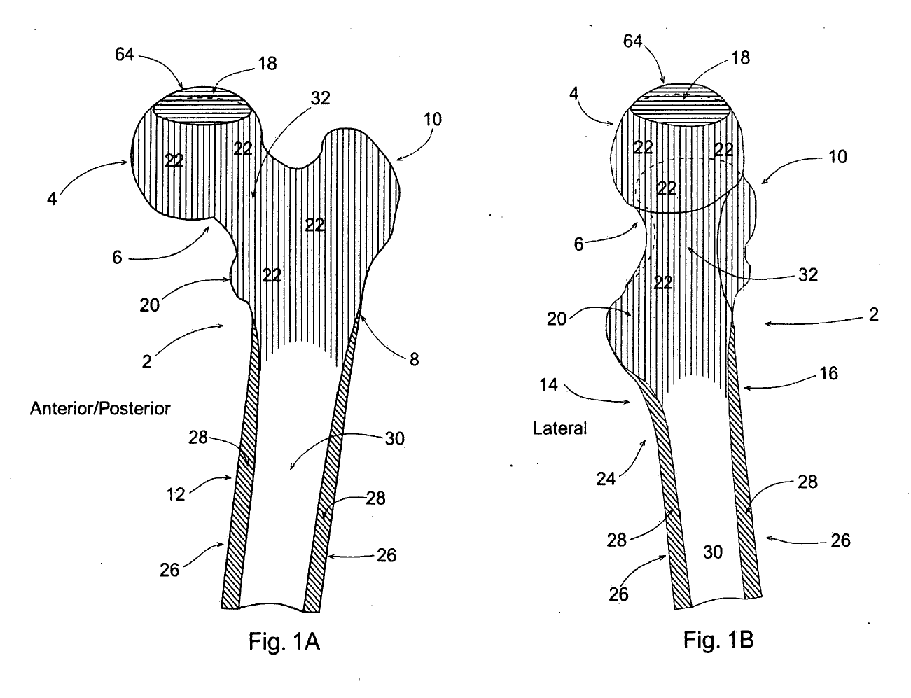

FIG. 1 shows a typical prior art surgical examination, illustrating in particular the number of incisions necessary for performing the surgical procedure. As suggested in FIG. 1, typical the instruments are arranged in a triangular orientation (triangulation) for observation, manipulation and evacuation of liberated debris. Triangulation in this manner, typically results in a number of users responsible for manipulation of the various surgical instruments being positioned in close proximity with each other. In addition, as illustrated in FIG. 1, a number of incisions are required to perform the triangulation procedure resulting in slower recovery and greater risk of undesired side effects such as infection.

FIGS. 1A and 1B generally refer to an anteroposterior view of a proximal femur having a femoral head 4 and a femoral neck 6 in anatomic confluency with a greater trochanter 11 and a lesser trochanter 20. Cancellous bone 22 is substantially confluent throughout the femoral head 4, the femoral neck 6 and the greater trochanter 11. In anatomic orientation described further herein, the femoral head 4 is situated proximally while the greater and lessor trochanters 11, 20 are situated distally. The femoral neck 6 establishes a cancellous bony pathway 32 therebetween the femoral head 4 proximally and the greater and lesser trochanters 11, 20 distally. The greater trochanter 11 and the lesser trochanter 20 are in anatomic confluency with a femoral shaft 26 having an outer radius R. The femoral shaft 26 is comprised of cortical bone 28 circumferential to a medullary canal 30 having an inner radius r and a neutral axis N. In the anteroposterior view of the proximal femur, the femoral shaft 26 includes a medial cortex 12 and a, lateral cortex 8. The medullary canal 30 establishes an areal moment of inertia I of the femoral shaft 26 to resist bending loads thereof to a substantial degree during normal gait. More specifically, the magnitude of compressive and tensile stresses during bending is defined as:

σ=My/I (2),

Where M is bending moment and is defined as the perpendicular distance from a line of force to a point of interest, y is the linear distance from the neutral axis, and I is the areal moment of inertia. The areal moment of inertia I is defined as follows:

I=¼π(R⁴−r⁴), (3).

Equation 3 shows that the areal moment of inertia I is inversely proportional to the magnitude of compressive or tensile stresses within the femoral shaft 26.

The areal moment of inertia for a thin wall cylinder is given by:

Iₜ=¼πr³t, (4),

where t is the thickness of the cylindrical wall.

In FIG. 1B, the proximal femur, the femoral shaft 26 having the medullary canal 30 includes a posterior cortex 14 and an anterior cortex 16. In addition, a segment of osteonecrotic bone 18 is shown and situated in the anterolateral portion of the femoral head 4.

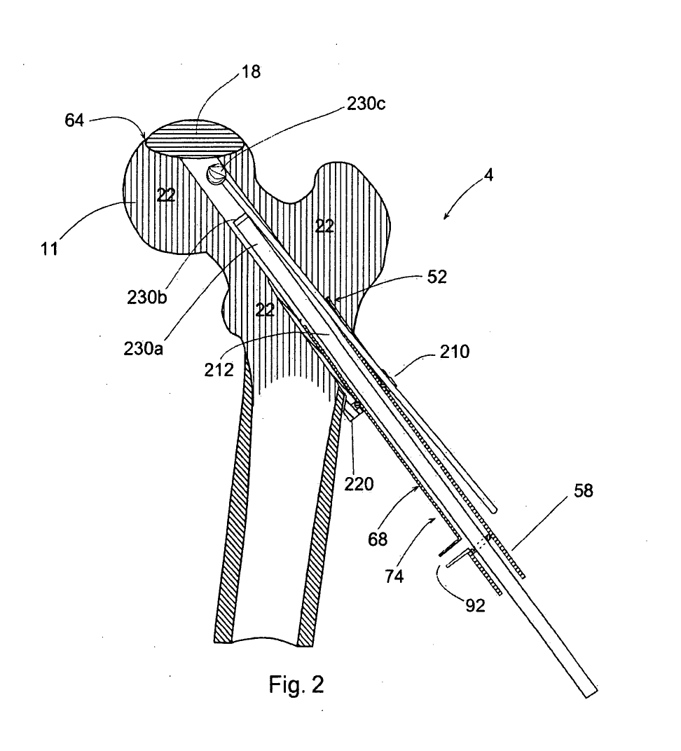

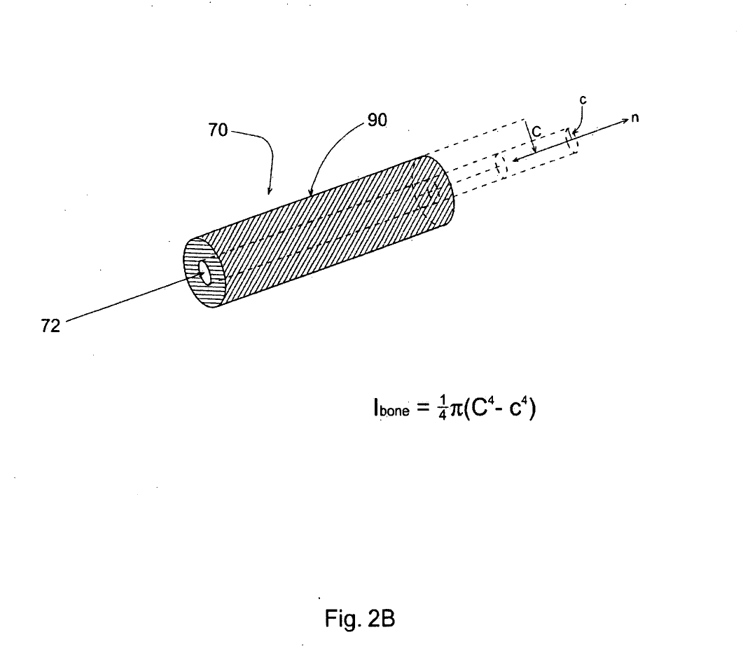

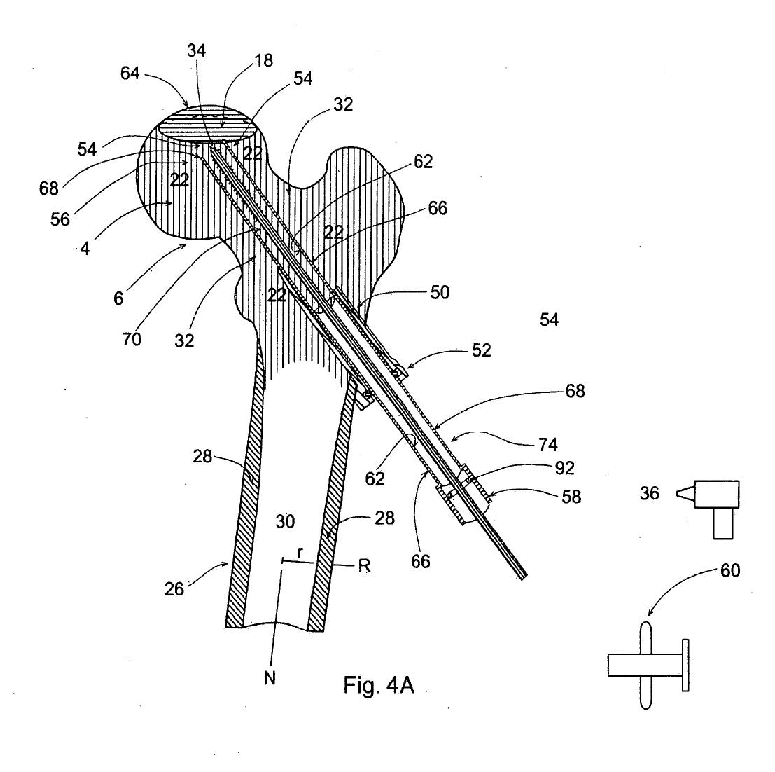

Referring to FIG. 2, an endoscopic bone debridement portal generally referred to herein by the reference numeral 210 is illustrated extending within the femoral head 4 and includes an outer surface presenting a hermetic seal at the juncture of the surrounding surface and the portal. In addition an endoscopic trephine having a distal endoscopic end, a proximal handle end, an inner visual surface and an outer contact surface is illustrated received by the endoscopic bone debridement portal 210. The endoscopic bone debridement portal 210 is further illustrated in receipt of plural surgical tools 230 such as, bit not limited to a visual device 230a e.g. an endoscopic video camera and a vacuum line 230b and a mechanical or electrical debridement tool 230c being advanced proximally through a lumen 212 or hollow region of the endoscopic bone debridement portal 230a. The surgical tools 230 are illustrated as being generally received by femoral head 4 from the endoscopic debridement portal 210.

The endoscopic bone debridement portal 210 of FIG. 2, is illustrated within the lateral portal of entry 50 having the cancellous bony pathway 32 antecedent thereto the segment of osteonecrotic bone 18. An ostomedullary cylinder 74 is passed through the endoscopic bone debridement portal 210 and advanced proximally into the femoral neck 6 and femoral head 4. The osteomedullary cylinder 74 passes through the cancellous bony pathway 32 to a first position 54 juxtainferior to the segment of osteonerotic bone 18. The osteomedullary cylinder 74 includes a distal bony end 56 and a proximal mechanical end 58 for mounting a handle 60 or the drill 36. The osteomedullary cylinder 74 further includes a first low friction inner surface 62 and a second low friction outer surface 66 in coaxial alignment so as to establish a material width 68 therebetween the inner and outer surfaces 62 and 66, respectively, of the osteomedullary cylinder 74. The material width 68 is of a dimension so as to establish a friction bony interface 80 therewithin the femoral neck 6.

FIG. 2B illustrates an autogenous cancellous osteomedullary bone cylinder 70 is uniformly porous having a length of at least 1 cm and an outer radius C and an osteoaxial canal 72 having an inner radius c from a neutral axius n is shown contained therewithin the osteomedullary cylinder 74. The plunger pin 34 is removed from the autogenous cancellous osteomedullary bone cylinder 70 by advancing the plunger pin 32 proximally thereabout the proximal mechanical end 58. In this regard, further shown proximally thereabout the proximal mechanical end 58 of the osteomedullary cylinder 74 is a proximal retaining support 92 of a size and shape adapted to prevent the removal of the autogenous cancellous osteomedullary bone cylinder 70 from the osteomedullary cylinder 74 in a proximal direction. The proximal retaining support 92 being circumferentially situated thereabout the proximal mechanical end 58 is further of a size and dimension adapted to allow a displacement plunger 82 to pass therethrough in a distal direction and into the osteomedullary cylinder 74.

More specifically, the autogenous cancellous osteomedullary bone cylinder 70 is only advanced into a proximal portion of the femoral head 4 and the femoral neck 6 by removal of the autogenous cancellous osteomedullary bone cylinder 70 from the osteomedullary cylinder 74 in a distal direction through the distal bony end 56. Therefore, the areal moment of interia I bone of the autogenous cancellous osteomedullary bone cylinder 70 is defined as:

I_{bone}=¼π(C⁴−c⁴) (5),

Wherein the geometric configuration thereof is of a construct to resist bending loads during normal gait as in Equation 2 above wherein the areal moment of inertia I_{bone} is inversely proportional to the magnitude of compressive or tensile stresses. More importantly, Equation 2 does not reflect the material properties of bone as does Young's Modulus. Young's modulus of elasticity for cancellous bone 22 may vary from approximately 10 MPa to 2,000 MPa, whereas that for cortical bone is approximately 17,000 MPa. In the geometric construct of the autogenous cancellous osteomedullary bone cylinder 70 of the present invention, the osteoaxial canal 72 substantially functions to decompress the femoral head 4 of increased intraosseous hydrostatic pressure characteristic of osteonecrosis of the femoral head 4.

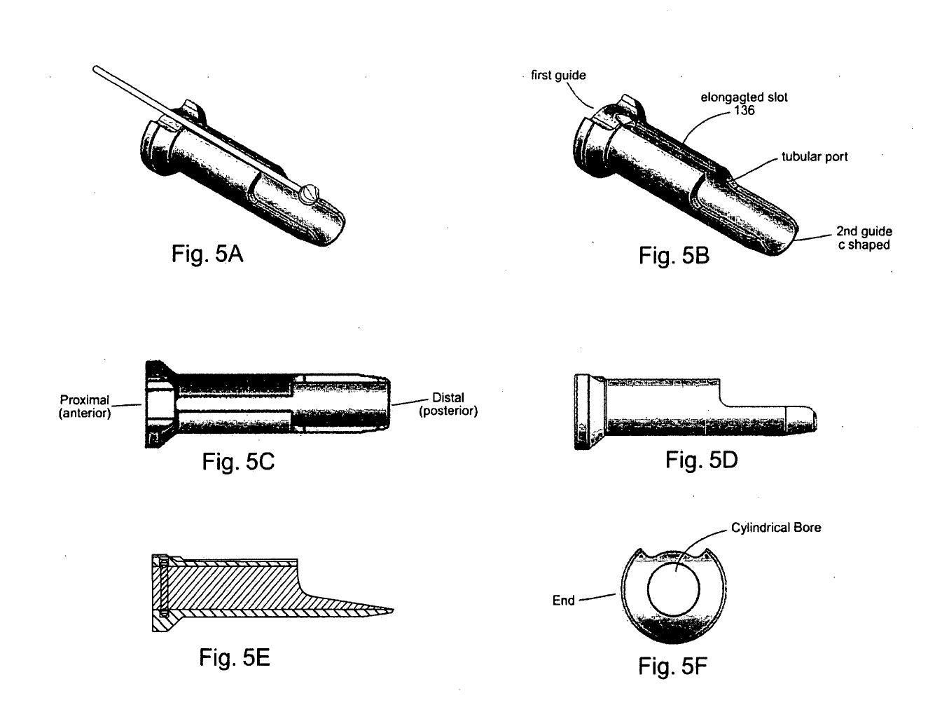

As further illustrated in FIGS. 5A-5F, the endoscopic debridement portal 210 generally includes an elongated sleeve 214 extending longitudinally between a medial incision 216 and a lateral surface 218 and presenting a central channel 222. In use, the endoscopic debridement portal 210 generally provides repeated entrance to a guided passageway between the incision to the surgical site, the elongated sleeve 214 extending subcutaneously with the lateral surface 218 being generally associated with a surgical site while the medial incision 216 is generally associated with the incision.

The elongated sleeve 214 presents the central channel 222 and circumferentially extends around a lumen 212 associated with the central channel 222 and is generally configured with plural guided surfaces adapted for receiving surgical tools or instruments having an operational head extending from a leader, the guided surfaces adapted for presenting the surgical tools in a centralized arrangement. The various guide surfaces associated with the endoscopic bone debridement portal 210 aligns various surgical tools 230 to be centrally aligned from the incision towards the surgical site in a central configuration.

As further illustrated in FIG. 5B, the endoscopic bone debridement portal 210 includes an inner surface and two integral guide surfaces, a first, wedge-shaped guide 224 and a second, C-shaped channel 226 extending towards the lateral surface 218. The wedge-shaped guide 224 is illustrated in FIG. 5C positioned along the outer circumference of the elongated sleeve 214 and extends inwardly towards the central channel 222, the wedge-shaped guide 224 being positioned superior to the C-shaped channel 226 which is inferior thereto.

The first and second shaped guide surfaces 224, 226 present several possible advantages, including the presentation and alignment of various surgical tools 230 at the surgical site. Particularly, the wedge-shaped guide 224, illustrated in FIGS. 5A-5F, facilitates slidable engagement with the surgical tool 230 such as a cutting device to debride any soft or hard materials such as tissue or bone. As illustrated in FIG. 5A, the cutting device 232 may be associated with one end of a rigid shaft 234. The wedge-shaped guide 224 is configured with an opening slightly larger than the outer diameter of the cutting device 232 so as to facilitate slidable cooperation therebetween with a minimal amount of free play. As the shaft 234 associated with the cutting device 232 is longitudinally extended from the incision, along the elongated sleeve 214 and towards the surgical site, the cutting device 232 may be angled. The degree of angularity will depend at least in part on the configuration of the wedge-shaped guide 224.

By receiving plural surgical tools 230 through an incision and extended the tools 230 longitudinally along the elongated sleeve 214, at least two guide surfaces allow the tools 230 to be aligned into a central or singular arrangement. This method of alignment of the surgical instruments into a singular arrangement, generally referred to as a singulation procedure allows the surgeon to utilize a single incision to position both the visual device 230a and surgical tool 230 during surgery to addressing the surgical site. Depending on the desired visual device 230a and desired surgical tool 230, the elongated sleeve 214 may have various possible configurations and sizes. In this way the guides could be efficiently and effectively sized and shaped to mimic the size and shape of the received endoscopic surgical tools.

Alternatively, as illustrated in FIGS. 5B and 5C, an elongated slot 236 may be utilized for provide greater angular freedom for positioning the cutting device near the surgical site.

The C-shaped guide 226 illustrated in FIG. 5B, presents a shaped edge 228 which for align the received visual devices 230a, surgical tools 230, and for allowing the surgeon to resect or otherwise limit any visual interference caused by any damaged or obstructing material.

The central channel 222, or lumen 212 is generally positioned circumferentially along the interior of the elongated sleeve 214 and extends between the wedge-shaped guide 224 and the C-shaped channel 226, allowing for visualization of the surgical site providing enhanced visual inspection and access to the surgical site.

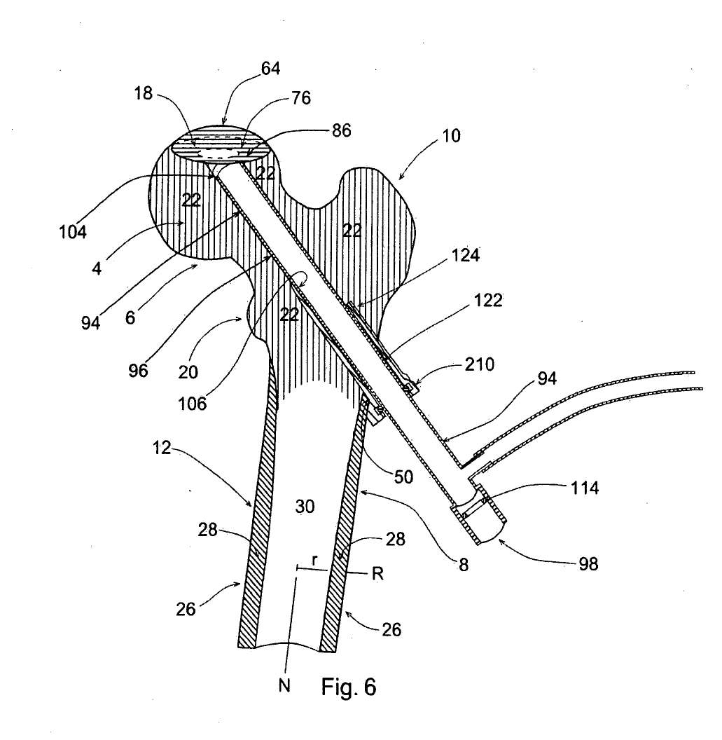

Turning now to FIG. 6 there shown is the osteoendoscopic cylinder 94 having been inserted into an osteocentral canal 100. The osteoendoscopic cylinder 94 includes an outer bony contact surface 96 of a dimension adapted to contact the longitudinal canal surface 102 so as to tamponade bleeding therefrom, and an inner visual surface 106. The outer bony contact surface 96 is further of a size and dimension adapted to establish a hermetic seal at the juncture thereof and the longitudinal canal surface 102.

A hermetic seal is also desirable at the juncture of the endoscopic bone debridement portal 210 and an inner bony surface 124 of the lateral portal of entry 50, and yet another hermetic seal at the juncture of the outer bony contact surface 96 and an inner centralizing surface 122 of the endoscopic bone debridement portal 210. The osteoendoscopic cylinder is of a size and dimension adapted to receive an endoscope coaxially along the inner visual surface and further includes a proximal handle end 98 and is shown having been engaged by the handle 60. A distal endoscopic end 104 is shown in proximity to the segment of osteonecrotic bone 18. Operationally situated thereabout the proximal handle end is a side opening 112 of a size and shape adapted to mount a vacuum apparatus 128 including a transparent tube adapted for evacuating a quantity of osteonecrotic bone fragments 108 debrided from the femoral head 4. The vacuum apparatus 128 induces a low pressure environment within the femoral head 4 so as to decompress an elevated intraosseous pressure therein. Further, the vacuum apparatus 128 induces blood to flow from the cancellous bone 22.

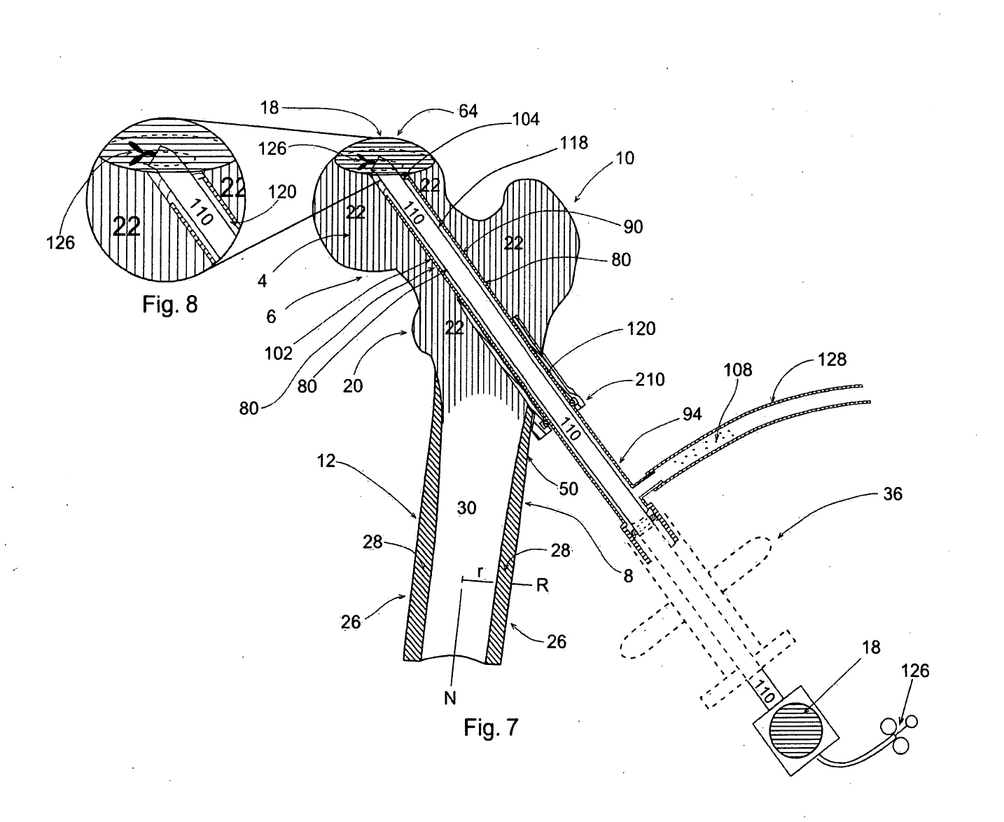

The distal endoscopic end 104 positioned within the femoral head 4, is illustrated in FIG. 7 situated juxtainferior to the segment of osteonecrotic bone 18. An endoscope 110 having a longitudinal material surface 118 is shown having been advanced into the osteoendoscopic cylinder substantially coaxially along the inner visual surface 106 as to create a bony particle chamber 120 for collecting the quantity of osteonecrotic bone fragments 108 debrided from the femoral head. The endoscope 110 passes into the osteoendoscopic cylinder 94 after first passing through the handle 60 and over a proximal stabilizing support 114, being of a size and dimension to allow distal and proximal advancement of the endoscope 110 within the osteoendoscopic cylinder 94. The proximal stabilizing support 114 is further of a size and dimension adapted to prevent the flow of air at the juncture thereof and the longitudinal material surface 118 of the endoscope 110. Now with the endoscope 110 within the osteoendoscopic cylinder 94, the surgeon may manipulate the optics thereof so as to visually observe the osteonecrotic bone 18 and the cancellous bone 22 within the femoral head 4.

FIG. 8 is an magnified sectional view of the proximal femur wherein a grasping instrument 126, a reamer, or a probe may be passed through the endoscope 110 and into the femoral head 4 as shown and in so doing, the femoral head 4 may be debrided under direct visualization. Debridement of the femoral head 4 creates an osteocavity 76 having a region of overlying cartilage 64 as is generally shown in FIG. 9. The osteocavity 76 is in structural confluency with the osteocentral canal 100.

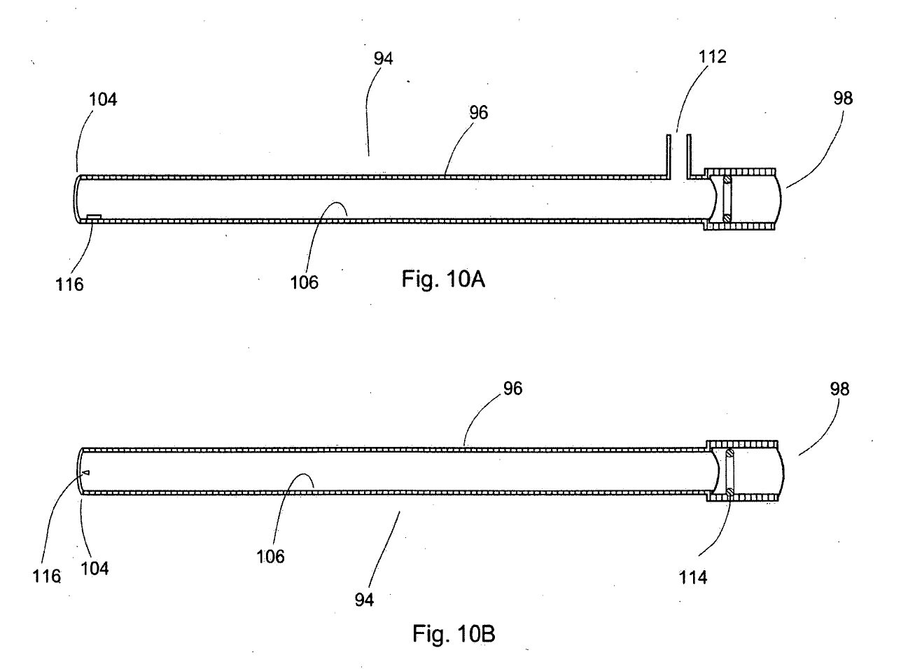

Turning now to FIGS. 10A and 10D, situated thereabout the distal endoscopic end 104 and along the inner visual surface 106 is an orientation mark 116. The orientation mark 116 is of a size and shape adapted to ensure a first visualization thereof with the endoscope 110. The orientation mark is in orthogonal alignment with the side opening 112 so as to ensure operational and spatial orientation with respect to the superior, inferor, anterior and posterior bony quadrants within the femoral head 4 at all times. More specifically, the surgeon may position the side opening 112 in a posterior direction and thereby position the orientation mark 116 anteriorly within the anterior bony quadrant of the femoral head 4. In this regard, debridement of the femoral head 4 is strategic in that the quality and the location of the osteonecrotic bone 18 then debrided under direct endoscopic visualization can be fully described.

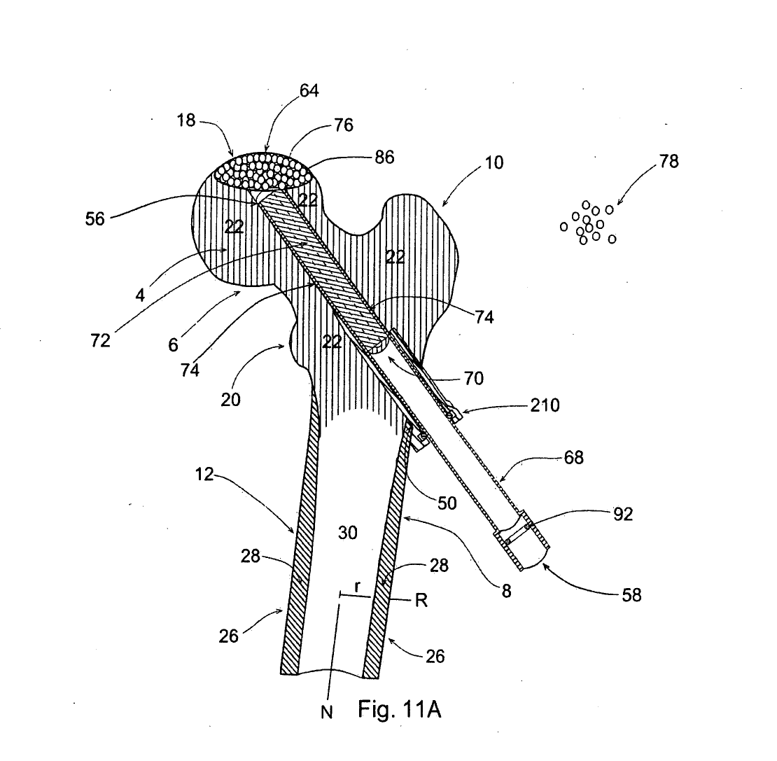

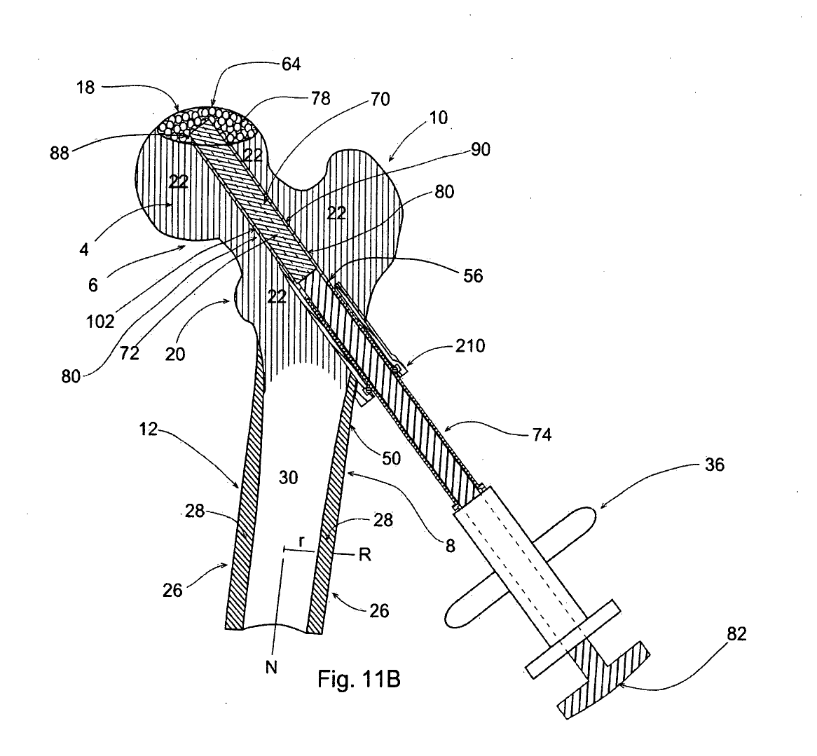

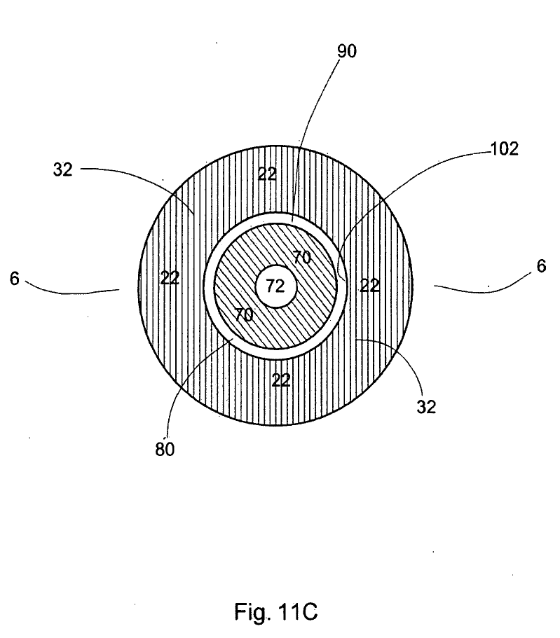

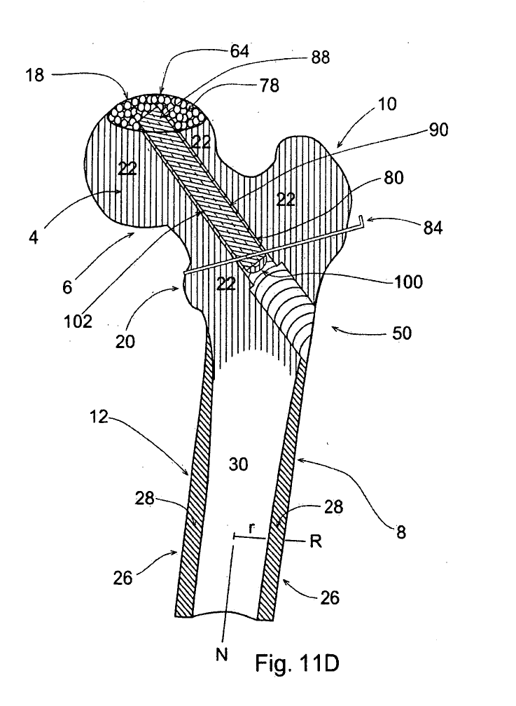

FIGS. 11A-11D illustrates the receipt of morselized cortical or cancellous bone graft 78 into the femoral head 4. The packing of the femoral head 4 illustrated in FIG. 11B, or replacement bone grafting as it may be called, is to a degree to completely fill the osteocavity 76 and to provide preliminary axial support to the region of overlying cartilage 64. FIG. 11A shows the autogenous cancellous osteomedullary bone cylinder 70 having been returned to a second position 86 juxtainferior to the now filled osteocavity 76. The osteoaxial canal 72 is in view. FIG. 11B shows the displacement plunger 82 having been passed through the osteomedullary cylinder 74 to further advance the autogenous cancellous osteomedullary bone cylinder 70 distally into a proximal portion of the femoral head 4 to a third position 88 juxtainferior to the region of overlying cartilage 64 so as to provide mechanical support thereto.

Importantly, the autogenous cancellous osteomedullary bone cylinder 70 is of a length and dimension to provide mechanical support to the region of overlying cartilage and simultaneously remains in contact with the longitudinal canal surface 102 of the osteocentral canal 100. A stabilizing wire 84 is shown transfixing the autogenous cancellous osteomedullary bone cylinder 70 so as to ensure the autogenous cancellous osteomedullary bone cylinder 70 remains in the third position 88. A friction bony interface or a stable cylindrical fracture 80 is shown circumferentially situated to the autogenous cancellous osteomedullary bone cylinder 70 and is established therebetween a longitudinal bony friction surface 90 of the autogenous cancellous osteomedullary bone cylinder 70 and the longitudinal canal surface 102 of the osteocentral canal 100.

FIG. 11C is a sagittal plane cross sectional view of the femoral neck 6 having the autogenous cancellous osteomedullary bone cylinder 70 centrally positioned therein. FIG. 11D shows the completed procedure wherein the osteomedullary cylinder 74, the displacement plunger 82, and the endoscopic debridement portal 110 have been removed.

In operation, and referring back to FIG. 2 in general, the endoscopic bone debridement portal 210 may be inserted, similarly to a centralizing sleeve (not shown), through an incision with the lateral surface 218 of the elongated sleeve 214 being positioned near the surgical site. The visual device 230a may then be inserted through the incision and received by the central channel and longitudinally extended along the C-shaped channel associated with the lateral surface 218. The cutting device 232, fixed to one end of the rigid shaft 234 may be inserted through the wedge-shaped guide and extended longitudinally along the elongated sleeve 214 towards the surgical site for preparation of the surgical site while the visual device 230a is utilized for observation from the same incision position.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

Having thus described the invention, what is claimed as new and desired to be secured by Letters Patent is as follows:

1. An endoscopic bone debridement portal adapted for use with surgical instruments extending between an incision and a surgical site, said endoscopic bone debridement portal comprising:
an elongated sleeve presenting a central channel and extending between a medial incision associated with the incision and a lateral surface associated with the surgical site;
said elongated sleeve having a first and a second guide surface, said first guide surface spaced radially from said second guide surface, said first and second guide surfaces adapted for centrally aligning the surgical tools, and
said central channel extending circumferentially around a lumen and between the plural guide surfaces whereby the surgical tools extend through the lumen from the incision towards the surgical site.

2. An endoscopic bone debridement portal in combination with an endoscopic trephine and plural surgical instruments extending between an incision and a surgical site, said combination comprising:
plural surgical instruments having an operational head extending from a leader said operational head adapted for surgical procedures,
an endoscopic trephine having a distal endoscopic end, a proximal handle end, an inner visual surface and an outer contact surface, said proximal handle opposite said distal endoscopic end and said inner visual surface associated with said distal endoscopic end along said outer contact surface,
an endoscopic portal having an inner surface and an outer surface presenting a seal at the juncture of the surrounding surface and the endoscopic portal,
said inner surface adapted for sealable receipt of the outer contact surface,
said distal endoscopic end adapted for passage through the endoscopic portal, and
said endoscopic portal having plural guide surfaces for centrally aligning plural surgical tools with said operational heads associated with the surgical site, said leaders positioned longitudinally through an incision opening adapted for receiving said portal.

3. The combination according to claim 2 wherein said endoscopic portal presents an elongated sleeve presenting a central channel and extending between a medial incision associated with the incision and a lateral surface associated with the surgical site.

4. The combination according to claim 2 wherein said central channel extends circumferentially around a lumen, between the plural guide surfaces for alignment of the surgical tools extending through the lumen from the incision towards the surgical site.

5. The combination according to claim 2 wherein said elongated sleeve is associated with said inner surface for centrally aligning the plural surgical instruments.

6. The combination according to claim 2 wherein said endoscopic trephine is adapted to communicate with a vacuum apparatus.
